(19) United States
(12) Patent Application Publication
(10) Pub. No.: US 2025/0154128 A1
(43) Pub. Date: May 15, 2025

(54) A PROCESS FOR MAKING OSIMERTINIB

(71) Applicant: Synthon B.V., Nijmegen (NL)

(72) Inventors: Libor VYKLICKY, Blansko (CZ); Miroslav ZABADAL, Blansko (CZ)

(21) Appl. No.: 18/686,210

(22) PCT Filed: Sep. 1, 2022
(86) PCT No.: PCT/EP2022/074288

§ 371 Date: Feb. 23, 2024

(30) Foreign Application Priority Data
Sep. 2, 2021 (EP) 21194510.0

(51) Int. Cl. C07D 403/04

(52) U.S. Cl. CPC C07D 403/04

(57) ABSTRACT

The invention deals with a novel process for preparation of the pharmaceutically useful product Osimertinib of formula (1).

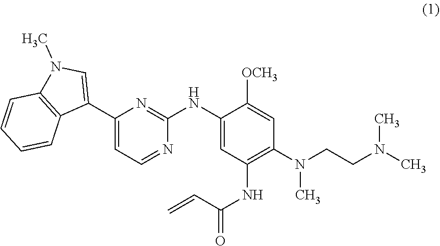 [SMILES: C=CC(=O)Nc1cc(Nc2nccc(-c3cn(C)c4ccccc34)n2)c(OC)cc1N(C)CCN(C)C]

The present invention relates to an improved process for preparation of osimertinib or a salt thereof.

### BACKGROUND OF THE INVENTION

Osimertinib, chemically N-(2-[[2-(Dimethylamino)ethyl](methyl)amino]-4-methoxy-5-[[4-(1-methyl-1H-indol-3-yl)pyrimidin-2-yl]amino]phenyl)acrylamide of formula (1),

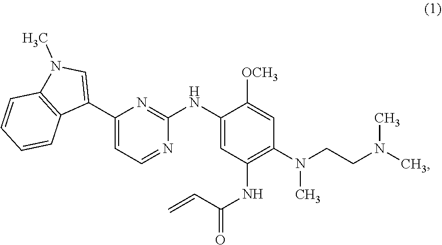 [SMILES: C=CC(=O)Nc1cc(Nc2nccc(-c3cn(C)c4ccccc34)n2)c(OC)cc1N(C)CCN(C)C]

is EGFR inhibitor, which is used for the treatment of advanced non-small cell lung cancer (NSCLC). Osimertinib is marketed as methanesuflonate salt under trade name Tagrisso by AstraZeneca. Osimertinib was first disclosed in WO2013014448. Several processes for preparation of Osimertinib are described in the prior art. The key step in Osimetrinib preparation is reduction of compound of formula (2) to prepare compound of formula (3):

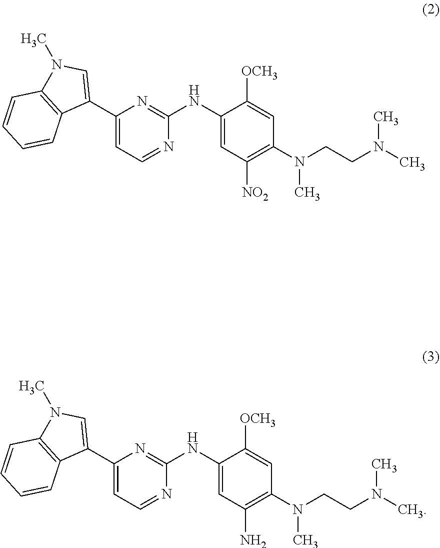 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

The process described in WO2013014448 application uses iron in conjunction with NH₄Cl for reduction of compound of formula (2) into compound of formula (3). This reduction is not suitable for large scale production since purification of the product requires ion exchange chromatography. WO2017134051 application describes a process for preparing compound of formula (3) by reduction of compound of formula (2) by using gaseous H₂ in pressure equipment (autoclave) in a presence of an acid. Then the stoichiometric amount of the acid needs to be neutralized by NaOH and removed thereby generating additional waste. Additionally to that using of gaseous H₂ at high pressure can represent a safety hazard.

Thus, there is still a need to find an improved process for preparation of Osimertinib and its key intermediate, compound of formula (3), which is suitable for high scale production and does not use hazardous gaseous H₂.

### BRIEF DESCRIPTION OF THE PRESENT INVENTION

The presented invention relates to a process for preparing compound of formula (1) or a salt thereof,

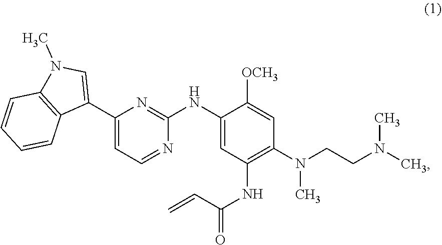 [SMILES: C=CC(=O)Nc1cc(Nc2nccc(-c3cn(C)c4ccccc34)n2)c(OC)cc1N(C)CCN(C)C]

comprising:
a. Reacting compound of formula (2) with hydrazine or ammonium formate in a presence of a catalyst in a solvent to obtain compound of formula (3):

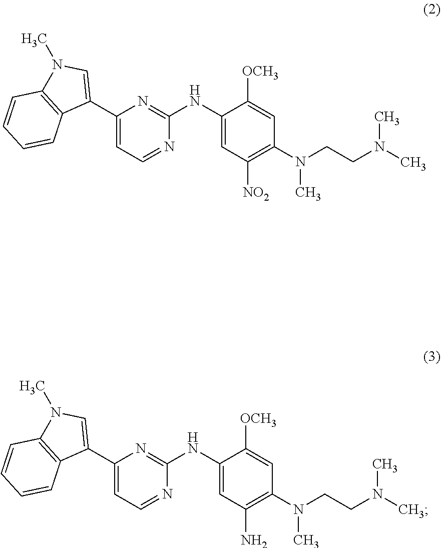 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

b. Transforming compound of formula (3) into compound of formula (1) or a salt thereof.

### DETAILED DESCRIPTION OF THE INVENTION

The presented invention relates to a process for preparing compound of formula (1) or a salt thereof,

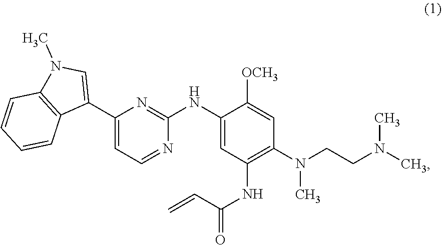 [SMILES: C=CC(=O)Nc1cc(Nc2nccc(-c3cn(C)c4ccccc34)n2)c(OC)cc1N(C)CCN(C)C]

comprising:
a. Reacting compound of formula (2) with hydrazine or ammonium formate in a presence of a catalyst in a solvent to obtain compound of formula (3),

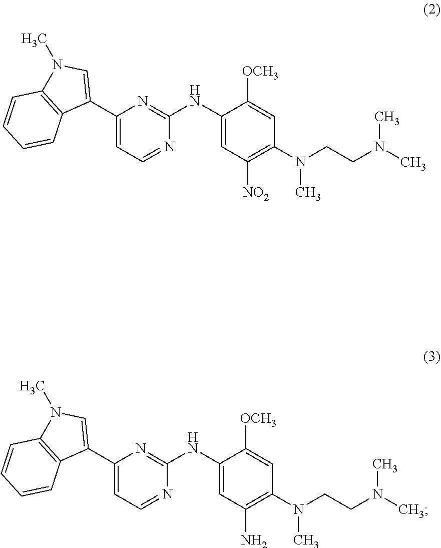 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

b. Transforming compound of formula (3) into compound of formula (1) or a salt thereof.

The catalyst in reaction step a. can be selected from Pd or Pt or Fe or Ni or Ru or Rh, preferably it is selected from Pd or Pt or Fe, more preferably Pd(0) on carbon (Pd/C) or Pt(0) on carbon (Pt/C) is used. In a preferred embodiment the catalyst, preferably Pd/C or Pt/C in concentration of 5-10% is used. The reaction step a. can be performed in a solvent, for example an ether such as dioxan or tetrahydrofuran or 2-methyl tetrahydrofuran or an alcohol such as methanol or ethanol or propanol or dimethylformamide or water, preferably 2-methyl tetrahydrofurane is used. The concentration of compound of formula (2) in the solvent can be between 0.04 g/ml and 0.1 g/ml, preferably it is between 0.05 g/ml and 0.08 g/ml. The molar ratio between the compound of formula (2) and hydrazine can be between 1:3 and 1:20, preferably it is between 1:10 and 1:15, more preferably it is between 1:11 and 1:13. The molar ratio between the compound of formula (2) and ammonium formate can be between 1:3 and 1:20, preferably it is between 1:10 and 1:15, more preferably it is between 1:11 and 1:13. The molar ratio between the compound of formula (2) and the catalyst can be between 20:1 and 2000:1, preferably it is between 50:1 and 200:1, more preferably it is between 195:1 and 200:1.

The reaction steps are preferably performed under a protective atmosphere, for example under nitrogen or argon.

Compound of formula (2) is mixed with the solvent. To the mixture the catalyst is added. The mixture is heated to a temperature between 60° C. and 90° C. To the mixture hydrazine, preferably hydrazine hydrate, or ammonium formate is added. The mixture is stirred at 60° C.-90° C. temperature for between 10 and 25 hours. The reaction progress can be monitored by any suitable analytical method for example by HPLC or GC.

When the reaction is finished, the mixture is cooled to a temperature between 20° C. and 25° C. To the mixture water is added. Volume ratio between added water and the solvent used in step a. can be between 1:0.5 and 1:1, preferably it is between 1:0.6 and 1:0.8. The mixture is stirred for between 5 and 10 minutes. The mixture is filtered off and the layers are separated. The organic layer is washed with water and the layers are separated. The washing step with water can be repeated for example 2× or 3× or 4× or 5×. The organic layer can be washed with brine and the layers are separated. The organic layer is then dried, for example by MgSO₄ and evaporated to provide compound of formula (3).

A solid form of compound of formula (3) can be prepared by a process comprising:
a. Dissolving compound of formula (3) in 2-methyl tetrahydrofurane;
b. Adding methyl tert-butyl ether;
c. Isolating the solid form of compound of formula (3).

The concentration of compound of formula (2) in 2-methyl tetrahydrofuran can be between 0.005 g/ml and 0.5 g/ml, preferably it is between 0.1 g/ml and 0.3 g/ml. Compound of formula (3) is dissolved in 2-methyl tetrahydrofuran optionally at an elevated temperature for example between 4° and 80° C. To the solution methyl tert-butyl ether is added. Volume ratio between 2-methyl tetrahydrofurane and methyl tert-butyl ether can be between 2:1 and 3:1, preferably it is between 2.2:1 and 2.6:1. The mixture is stirred for between 1.5 and 4 hours. Obtained solid is filtered off and can be optionally washed with methyl tert-butyl ether.

Compound of formula (3) can be transformed into compound of formula (1) or a salt thereof by a process known in the prior art or by a process comprising:
a. Reacting compound of formula (3) with acryloyl halide (compound of formula 4) in a solvent to provide compound of formula (1),

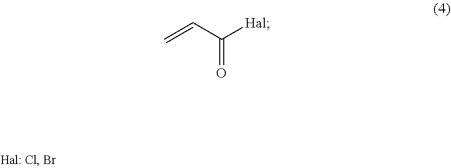

b. Optionally converting compound of formula (1) into a salt;
or
a. Reacting compound of formula (3) with 3-halopropanoyl halide (compound of formula 5) in a solvent to obtain compound of formula (6);

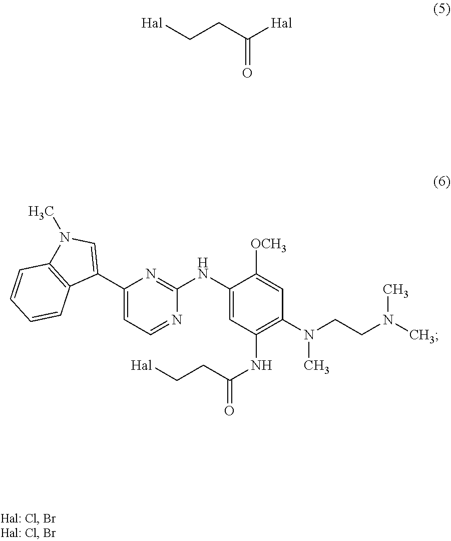

b. Converting compound of formula (6) into compound of formula (1);
c. Optionally converting compound of formula (1) into a salt thereof.

In the case when compound of formula (3) is reacted with acryloyl halide (compound of formula 4), the concentration of compound of formula (3) in the solvent can be between 0.02 g/ml and 0.07 g/ml, preferably it is between 0.04 g/ml and 0.06 g/ml. The solvent can be selected from dimethylformamide or a chlorinated solvent such as dichloromethane or trichloromethane or tetrachloromethane or an ether such as dioxane or 2-methyl tetrahydrofuran or tetrahydrofuran, preferably it is 2-methyl tetrahydrofuran. The molar ratio between compound of formula (3) and acryloyl halide (compound of formula 4) can be between 1:1 and 1:2, preferably it is between 1:1.3 and 1:1.5. The reaction can be performed in a presence of a base. As base for example an inorganic base such as a carbonate such as sodium carbonate or potassium carbonate or a hydrogen carbonate such as sodium hydrogen carbonate or potassium hydrogen carbonate or a hydroxide such as sodium hydroxide or potassium hydroxide or an organic base such as triethylamine can be used. The molar ratio between the base and the compound of formula (3) can be between 1.5:1 and 2.2:1. Compound of formula (3) is mixed with the solvent, to the mixture the base is optionally added. The base can be optionally used in a form of a solution in a solvent, for example as water solution. To the mixture acryloyl halide in a course of between 1 and 10 minutes is added. The mixture is stirred at temperature between 0° C. and 30° C. for between 1 and 3 hours. The reaction progress can be monitored by any suitable analytical method for example by HPLC or GC. After the reaction is completed the layers are separated and the organic phase is washed for example with water or brine. The washing step can be repeated for example 2× or 3× or 4× or 5×. The organic phase is dried for example using MgSO₄, filtrated and concentrated to obtain compound of formula (1).

In the case when compound of formula (3) is reacted with 3-halopropanoyl halide (compound of formula 5), the concentration of compound of formula (3) in the solvent can be between 0.02 and 0.07 g/ml, preferably it is between 0.04 g/ml and 0.06 g/ml. The solvent can be selected from dimethylformamide or a chlorinated solvent such as dichloromethane or trichloromethane or tetrachloromethane or an ether such as dioxane or 2-methyl tetrahydrofuran or tetrahydrofuran, preferably it is 2-methyl tetrahydrofuran. The molar ratio between compound of formula (3) and 3-halopropanoyl halide can be between 1:1 and 1:3, preferably it is between 1:2.5 and 1:2.7. The reaction can be performed in a presence of a base. As base for example an inorganic base such as a carbonate such as sodium carbonate or potassium carbonate or a hydrogen carbonate such as sodium hydrogen carbonate or potassium hydrogen carbonate or a hydroxide such as sodium hydroxide or potassium hydroxide or an organic base such as triethylamine can be used. The molar ratio between the base and the compound of formula (3) can be between 1.5:1 and 2.2:1. Compound of formula (3) is mixed with the solvent, to the mixture the base is optionally added. The base can be optionally used in a form of a solution in a solvent, for example as water solution. To the mixture 3-halopropanoyl halide in a course of between 1 and 10 minutes is added. The mixture is stirred at temperature between 0° C. and 30° C. for between 1 and 3 hours. The reaction progress can be monitored by any suitable analytical method for example by HPLC or GC. After the reaction is completed the layers are separated and the organic phase is washed for example with water or brine. The washing step can be repeated for example 2× or 3× or 4× or 5×. The organic phase is dried for example using MgSO₄, filtrated and concentrated to obtain compound of formula (6). Compound of formula (6) can be transformed into compound of formula (1) by using a process described in the prior art, for example in WO2017134051 application.

Compound of formula (1) can be transformed into a salt thereof by reacting with a suitable acid, for example hydrochloric acid or hydrobromic acid or sulphuric acid or phosphoric acid or formic acid or acetic acid or trifluoroacetic acid or citric acid or maleic acid or oxalic acid or benzoic acid or fumaric acid or succinic acid or tartaric acid or lactic acid or pyruvic acid or methane sulfonic acid or ethane sulfonic acid or benzene sulfonic acid or p-toluene sulfonic acid, preferably with methane sulfonic acid, in a suitable solvent, for example acetonitrile or dimethylformamide or a chlorinated solvent such as dichloromethane or trichloromethane or tetrachloromethane or an ether such as dioxane or 2-methyl tetrahydrofuran or tetrahydrofuran, preferably in 2-methyl tetrahydrofuran.

Compound of formula (2) can be prepared by a process disclosed in the prior art or by a process comprising reacting compounds of formula (7) and (8) in a suitable solvent,

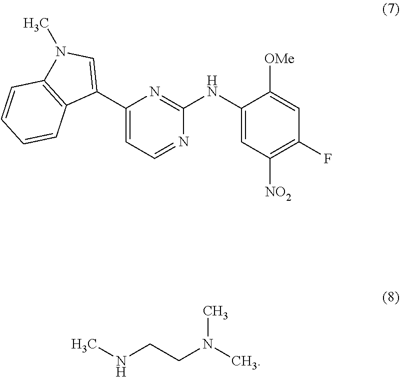

As a solvent for example dimethylformamide or a chlorinated solvent such as dichloromethane or trichloromethane or tetrachloromethane or an ether such as 1,4-dioxane or 2-methyl tetrahydrofuran or tetrahydrofuran, preferably it is 1,4-dioxane. The reaction is performed in a presence of sodium hydroxide and boric acid, at pH higher than 9. Concentration of compound of formula (7) in the solvent can be between 0.15 g/ml and 0.5 g/ml. The concentration of compound of formula (8) in the solvent can be between 0.05 g/ml and 0.1 g/ml. The molar ratio between compounds (7) and (8) can be between 1:1.3 and 1:1.7. Compound of formula (7) is mixed with the solvent and the buffer. To the mixture compound of formula (8) is added. The mixture is heated to a temperature between 100° C. and 110° C. and stirred at this temperature for between 5 and 12 hours. The reaction progress can be monitored by any suitable analytical method for example by HPLC or GC. After the reaction is completed, water and saturated water solution of a base are added. As base for example an inorganic base such as a carbonate such as sodium carbonate or potassium carbonate or a hydrogen carbonate such as sodium hydrogen carbonate or potassium hydrogen carbonate or a hydroxide such as sodium hydroxide or potassium hydroxide or an organic base such as triethylamine can be used. The volume ratio between added water and the solvent used for reaction of compounds (7) and (8) can be between 1:0.8 and 1:1.1. The volume ratio between added saturated water solution of the base and the solvent used for reaction of compounds (7) and (8) can be between 1:0.8 and 1:1.1. The mixture is heated to a temperature between 100° C. and 110° C. and stirred at this temperature for between 15 and 60 minutes. The mixture is cooled to a temperature between 20° C. and 25° C. and stirred at this temperature for between 5 and 15 hours to obtain a suspension. Obtained solid compound of formula (2) is filtered off and optionally washed with water and dried.

Compound of formula (8) is commercially available.

Compound of formula (7) can be prepared by a process disclosed in the prior art or by a process comprising reacting compounds of formula (9) and (10) in a suitable solvent,

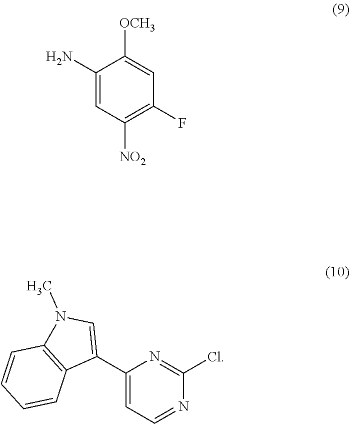 [SMILES: COc1cc(F)c([N+](=O)[O-])cc1N ; Cn1cc(-c2ccnc(Cl)n2)c2ccccc21]

The solvent can be selected for example from dimethylformamide or an ether such as 1,4-dioxane or 2-methyl tetrahydrofuran or tetrahydrofuran. The concentration of compound of formula (9) in the solvent can be between 0.05 g/ml and 0.1 g/ml. The concentration of compound of formula (10) in the solvent can be between 0.07 g/ml and 0.12 g/ml. The molar ratio between compounds (9) and (10) can be between 1:1.5 and 1:2.5, preferably it is between 1:1.5 and 1:2. The reaction is performed in a presence of an acid for example p-toluene sulfonic acid or methanesulfonic acid. The molar ratio between the acid and compound of formula (10) can be between 1:1 and 2:1.

Compounds of formula (9) and (10) are mixed with the solvent. The mixture is stirred for between 10 and 60 minutes and to the mixture the acid is added. The mixture is stirred for between 10 and 60 minutes. The mixture is heated to a temperature between 80° C. and the reflux temperature of used solvent for between 3 and 10 hours and then it is cooled to a temperature between 45° C. and 55° C. To the mixture a base, for example 25% water solution of ammonia or an organic amine such as dimethylamine or triethylamine is added unless pH of the mixture is higher than 8. The base is preferably added in portion, for example in 2 or 3 or 4 or 5 or 6 portions, more preferably it is added dropwise. The mixture is heated to a temperature between 80° C. and 90° C. and to the mixture water is added. The volume ratio between added water and the solvent used for reaction between compounds (9) and (10) can be between 1:2.5 and 1:3.5, preferably it is between 1:2.8 and 1:3.1. Water is preferably added in portion, for example in 2 or 3 or 4 or 5 or 6 portions, more preferably it is added dropwise. The mixture is stirred at a temperature between 20° C. and 25° C. for between 1 and 5 hours to obtain a suspension. Solid compound of formula (7) is filtered off and optionally washed with water or a mixture of acetonitrile and water and dried.

Compound of formula (9) is commercially available.

Compound of formula (10) can be prepared by a process disclosed in the prior art or by a process comprising reacting compounds of formula (11) and (12) in a suitable solvent,

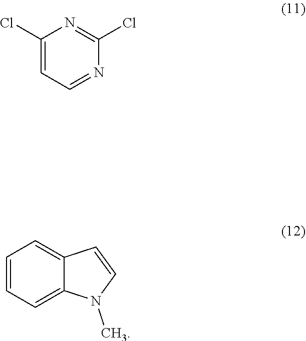 [SMILES: Clc1ccnc(Cl)n1 ; Cn1ccc2ccccc21]

As a solvent for example dimethoxyethane or dimethylformamide or a chlorinated solvent such as dichloromethane or trichloromethane or tetrachloromethane or an ether such as 1,4-dioxane or 2-methyl tetrahydrofuran or tetrahydrofuran, preferably dimethoxyethane is used. The concentration of compound of formula (11) in the solvent can be between 0.1 g/ml and 0.2 g/ml. The concentration of compound of formula (12) in the solvent can be between 0.1 g/ml and 0.2 g/ml. The molar ratio between compounds of formula (11) and (12) can be between 1:1 and 1:1.2. The reaction is performed in a presence AlCl₃. The molar ratio between compound of formula (11) and AlCl₃ can be between 1:1 and 1:1.3, preferably it is between 1:1 and 1:1.1.

Compound of formula (11) is mixed with the solvent and cooled to a temperature between −10° C. and 5° C. To the mixture AlCl₃ is added. AlCl₃ can be added in portions, for example in 2 or 3 or 4 or 5 or 6 portions. The temperature of the mixture is maintained at a temperature lower than 30° C. during the AlCl₃ addition. The mixture is then stirred at a temperature lower than 30° C. for between 20 and 60 minutes. To the mixture compound of formula (12) is added. The mixture is heated to a temperature between 70° C. and 90° C. and stirred at this temperature for between 2 and 5 hours. The reaction progress can be monitored by any suitable analytical method for example by HPLC or GC. After the reaction is completed the mixture is cooled to a temperature between 0° C. and 10° C. The mixture is added to water. The volume ratio between water and solvent used for reaction between compounds of formula (11) and (12) can be between 9:1 and 12:1. The mixture is stirred for between 1 and 6 hours. Solid compound of formula (10) is filtered off and optionally washed with water or a mixture of water and acetonitrile and dried. To improve the filterability of filtrated mixture a solution of an acid, for example water solution of HCl can be added to the filtrated mixture.

Solid compound of formula (10) can be purified by a process comprising:
a. Contacting compound of formula (10) with acetonitrile;
b. Heating the mixture to a temperature between 70° C. and the reflux temperature of the mixture;
c. Adding water, wherein the volume ratio between acetonitrile and water can be between 5:1 and 8:1, preferably between 6:1 and 7:1;
d. Isolating the solid compound of formula (10).

The concentration of compound of formula (10) in acetonitrile can be between 0.1 g/ml and 0.2 g/ml. Compound of formula (10) is mixed with acetonitrile. The mixture is heated to a temperature between 70° C. and reflux temperature of the mixture and stirred at this temperature for between 1.5 and 4 hours. To the mixture water is added, wherein the volume ratio between acetonitrile and water can be between 5:1 and 8:1, preferably between 6:1 and 7:1. The mixture is cooled to a temperature between 20° C. and 25° C. and stirred at this temperature for between 1 and 5 hours. The mixture is then cooled to a temperature between −10° C. and 10° C., preferably between 0° C. and 5° C. and stirred at this temperature for between 20 and 60 minutes. Obtained solid compound of formula (10) is filtered off and can be optionally washed with a mixture of acetonitrile and water, for example a mixture 70:30 (vol:vol) acetonitrile:water and dried.

The invention will be further described with reference to the following examples.

### EXAMPLES

### Example 1: Preparation of 3-(2-chloropyrimidin-4-yl)-1-methyl-1H-indole, Compound of Formula (10)

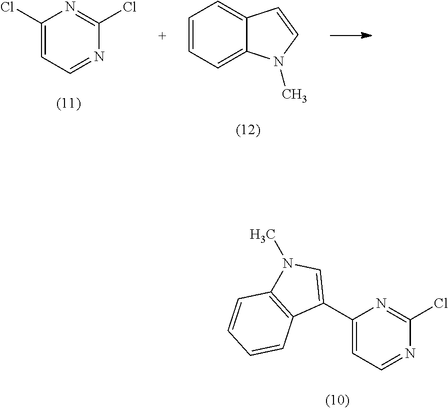

56.80 g of compound of formula (11) was dissolved in 400 ml of anhydrous dimethoxyethane and the solution was cooled to 0-5° C. Then 55.9 g of granulated AlCl₃ was added portionwise (6 portions during 17 minutes on ice bath) to a solution at the temperature below 30° C. and the suspension was stirred for 30 minutes. Then 50.0 g of compound of formula (12) was added, the suspension was stirred at 85° C. for 2 hours and then left to stand in the fridge (0-5° C.) for 12 hours.

The solution was poured into 4000 ml of water and the mixture was stirred for 1 hour. The solid was filtered off. To improve the filterability of the mixture, to the mixture 200 ml of 1M HCl can be added. Filtrated mass was washed with 500 ml of water and 500 ml of acetonitrile-water mixture (170:30). The solid was air dried to provide 76.57 g (82% of theoretical yield) compound of formula (10) in purity 93.6%.

### Example 2: Purification of Compound of Formula (10)

76.57 g of compound of formula (10) was mixed with 460 ml of acetonitrile. The mixture was heated to reflux and stirred at this temperature for 1 hour. To the mixture 69 ml of water was added at reflux. The mixture was allowed to cool to 20° C.-25° C. and stirred for 1 hour. Then the mixture was stirred at 5° C. for 30 minutes, the solid was filtered off and washed 4× with 25 ml of mixture of acetonitrile/water (70:30, vol:vol) and dried in vacuo at 50° C. to provide 71.17 g (77% of theoretical yield), 97% purity (HPLC(IN)).

### Example 3: Preparation of N-(4-fluoro-2-methoxy-5-nitrophenyl)-4-(1-methyl-1H-indol-3-yl)pyrimidin-2-amine (Compound of Formula (7))

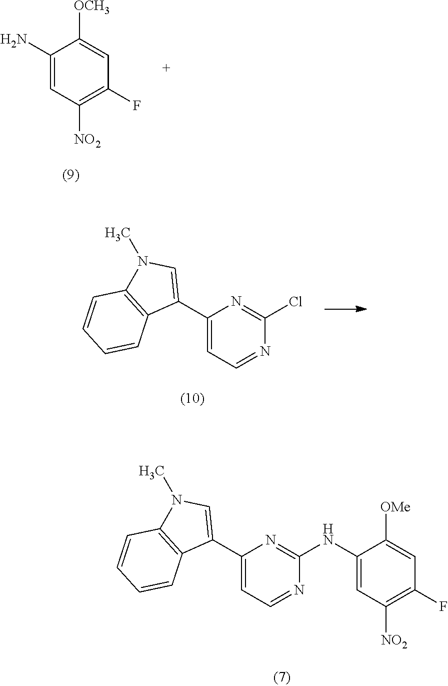 [SMILES: COc1cc(F)c([N+](=O)[O-])cc1N ; Cn1cc(-c2ccnc(Cl)n2)c2ccccc21 ; COc1cc(F)c([N+](=O)[O-])cc1Nc1nccc(-c2cn(C)c3ccccc23)n1]

Reaction was done under argon atmosphere. 25 g of compound of formula (10) and 19.10 g of compound of formula (9) were mixed with 290 ml of 1,4-dioxane. The suspension was stirred at 20° C.-25° C. for 15 minutes. Then 19.51 g of p-toluene sulfonic acid monohydrate was added and the suspension was stirred at 20-25° C. for 15 minutes. The suspension was stirred at 85° C. for 4 hours. The mixture was then heated under reflux (100° C.) for 30 minutes and then cooled to 50° C. during 1 h. 20 ml of 25% water solution of ammonia was added dropwise into the mixture during 15 min. Then the temperature was increased to 85° C. and 100 ml of water was added dropwise during 15 minutes. The suspension was stirred at 20° C.-25° C. for 1 hour. Product was filtered, washed with 2×100 ml of water and 100 ml of acetonitrile-water mixture (1:1, vol:vol). Product was dried at 100 mbar and 50° C. to provide 37.75 g (94% of theoretical yield) of solid compound of formula (7) in 99% purity HPLC(IN)).

### Example 4: Preparation of N¹-(2-(dimethylamino)ethyl)-5-methoxy-N¹-methyl-N⁴-(4-(1-methyl-1H-indol-3-yl)pyrimidin-2-yl)-2-nitrobenzene-1,4-diamine (Compound of Formula (2))

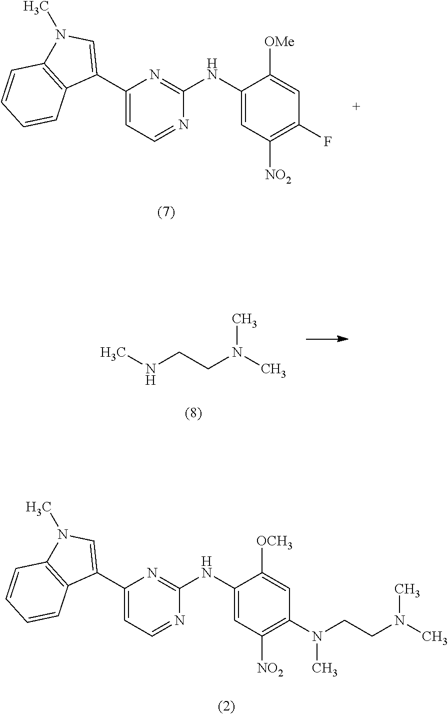

Reaction was done under argon atmosphere. 1.485 g of boric acid and 0.961 g of sodium hydroxide were dissolved in 17.50 ml of water. To the mixture 175 ml of 1,4-dioxane and 35 g of compound of formula (7) and 13.64 g of N¹,N¹,N²-trimethylethane-1,2-diamine (compound of formula (8)) were added. The mixture was stirred at 1000-105° C. (reflux) for 6 hours. To the mixture 175 ml of water and 1750 ml of saturated solution of NaHCO₃ were added at 105° C. and the mixture was stirred at 105° C. for 20 minutes. Then the mixture was cooled to 20° C.-25° C. during 1 hour and stirred at this temperature overnight. Product was filtered, washed with 3×100 ml of water and dried (100 mbar, 50° C.) to provide 41.696 g (99% of theoretical yield) of compound of formula (2), 94.6% purity (HPLC(IN)).

### Example 5: Preparation of N¹-(2-(dimethylamino)ethyl)-5-methoxy-N¹-methyl-N⁴-(4-(1-methyl-1H-indol-3-yl)pyrimidin-2-yl)benzene-1,2,4-triamine (Compound of Formula (3))

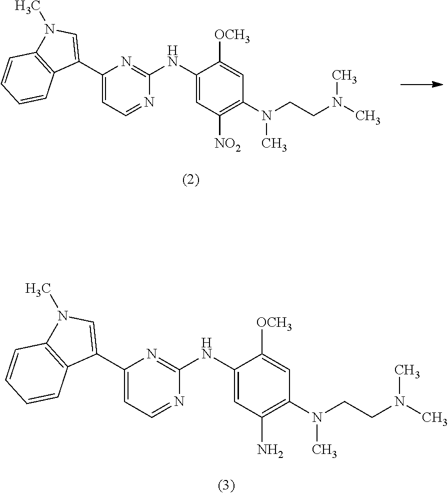 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

Reaction was done under argon atmosphere.

1 g of compound of formula (2) was mixed with 0.05 g of 5% Palladium on activated carbon (Pd/C). To the mixture 15 ml of 2-Methyltetrahydrofuran was added. The mixture was then heated to 85° C. 1.228 ml of hydrazine hydrate was added. The mixture was stirred for 20 hours and then was allowed to cool to 20° C.-25° C. To the mixture 10 ml of water was added. The mixture was filtered off. The layers were separated and the organic layer was washed 3× with water, 1× with brine, dried over MgSO₄ and evaporated. The rest was then dissolved in 6 ml of 2-Methyltetrahydrofuran by heating the mixture to temperature between 50-70° C. The mixture was cooled to 20° C.-25° C. and stirred at this temperature to start crystallization. After crystals started forming to the mixture 2.5 ml of methyl tert-butyl ether was added and the mixture was immersed in ice bath. After 2 hours the solid was filtered off and washed with 5 ml of methyl tert-butyl ether. The solid was then suction dried followed by vacuum drying at 40° C. for 1 hour to provide 0.803 g (79% of theoretical yield) of compound of formula (2), purity 94.1%.

### Example 6: Preparation of N¹-(2-(dimethylamino)ethyl)-5-methoxy-N¹-methyl-N⁴-(4-(1-methyl-1H-indol-3-yl)pyrimidin-2-yl)benzene-1,2,4-triamine (Compound of Formula (3))

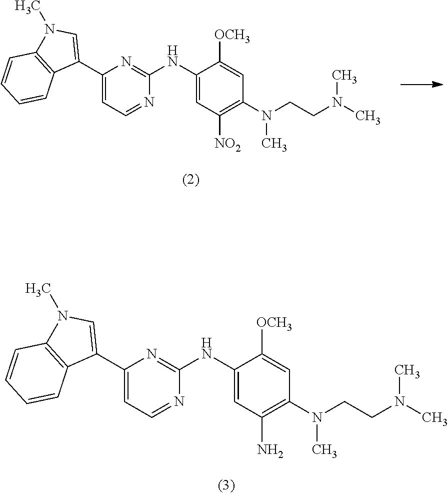 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

0.095 g of compound of formula (2), 0.003 g of Iron tri(4-methoxypent-3-en-2-one), 2 ml of methanol and 0.017 ml of hydrazine hydrate were charged into a 10 ml microwave vial. The mixture was then heated for 10 minutes at 150° C. in the micro wave reactor.

The reaction mixture was used to prepare compound of formula (1).

### Example 7: Preparation of N¹-(2-(dimethylamino)ethyl)-5-methoxy-N¹-methyl-N⁴-(4-(1-methyl-1H-indol-3-yl)pyrimidin-2-yl)benzene-1,2,4-triamine (Compound of Formula (3))

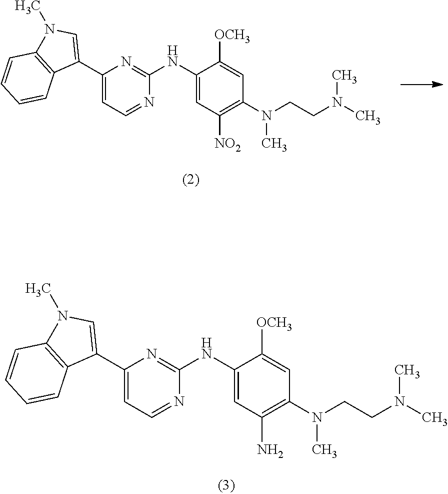 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

0.095 g of compound of formula (2), 0.003 g of Iron tri(4-methoxypent-3-en-2-one), 1.5 ml of methanol and 0.017 ml of hydrazine hydrate were mixed. The mixture was then heated to reflux (86° C. oil bath) for 72 hours. The mixture was allowed to cool to 20° C.-25° C.

Methanol was evaporated. 1.5 ml of 2-Methyltetrahydrofuran was added. The mixture was concentrated. The rest was dissolved in 1.5 ml of 2-Methyltetrahydrofuran. The mixture was used to prepare compound of formula (1).

### Example 8: Preparation of N¹-(2-(dimethylamino)ethyl)-5-methoxy-N¹-methyl-N⁴-(4-(1-methyl-1H-indol-3-yl)pyrimidin-2-yl)benzene-1,2,4-triamine (Compound of Formula (3))

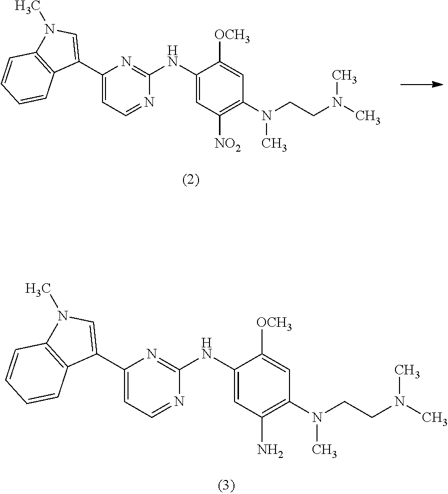 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

1 g of compound of formula (2) was combined with 0.05 g of 5% Palladium on activated carbon (Pd/C) and 1.59 g of ammonium formate. The solids were placed under inert atmosphere and 15 ml of 2-methyltetrahydrofuran was added. The reaction mixture was heated to reflux (85° C.) and stirred at this temperature for 23 hours.

Upon cooling to ambient temperature the catalyst was filtered off, the organic solution was washed 3× with 15 ml of water and then with 15 ml of brine, dried over MgSO₄ and concentrated to provide 6 g of a concentrated solution. The concentrated solution was stirred in ice bath for 2 hours and filtered. The filter cake was washed with 5 ml of methyl tert-butyl ether and vacuum dried at 40° C. for 1 hour to provide 0.72 g compound of formula (3) (68% yield) with purity 97.3%.

### Example 9: Preparation of N¹-(2-(dimethylamino)ethyl)-5-methoxy-N¹-methyl-N⁴-(4-(1-methyl-1H-indol-3-yl)pyrimidin-2-yl)benzene-1,2,4-triamine (Compound of Formula (3))

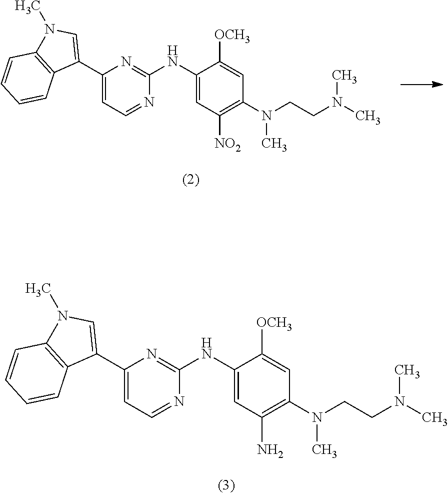 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]

Reaction was done under argon atmosphere.

1 g of compound of formula (2) was mixed with 0.05 g of 5% Pt/C. To the mixture 15 ml of 2-Methyltetrahydrofuran was added. The mixture was then heated to 85° C. 1.228 ml of hydrazine hydrate was added. The mixture was stirred for 20 hours and then was allowed to cool to 20° C.-25° C. To the mixture 10 ml of water was added. The mixture was filtered off.

The layers were separated and the organic layer was washed 3× with water, 1× with brine, dried over MgSO₄ and evaporated. The rest was then dissolved in 6 ml of 2-Methyltetrahydrofuran by heating the mixture to temperature between 50-70° C. The mixture was cooled to 20° C.-25° C. and stirred at this temperature to start crystallization. After crystals started forming to the mixture 2.5 ml of methyl tert-butyl ether was added and the mixture was immersed in ice bath. After 2 hours the solid was filtered off and washed with 5 ml of methyl tert-butyl ether. The solid was then suction dried followed by vacuum drying at 40° C. for 1 hour to provide 0.83 g (82% of theoretical yield) of compound of formula (2), purity 92%.

### Example 10: Comparative Example

The results of reduction of compound of formula (2) to provide compound of formula (3) according to presented invention were compared to result of a procedure disclosed in the prior art (Example 1 of WO2017134051).

| Catalyst | Isolated yield (%) | Purity (%) |
|---|---|---|
| Pt/C, H2, MsOH | Product was not isolated | The product was not |
| WO2017134051, | because of low reaction | isolated, the conversion |
| Example 1 | conversion | in the reaction |
| | | mixture was 60% |
| Pd/C, N2H4 | 79 | 94.1 |
| Example 5 of the | | |
| presented invention | | |
| Pd/C, HCOONH4 | 68 | 97.3 |
| Example 8 of the | | |
| presented invention | | |
| Pt/C, N2H4 | 82 | 92 |
| Example 9 of the | | |
| presented invention | | |

It can be concluded that using Pd/C or Pt/C in combination with hydrazine or ammonium formate provides compound of formula (3) in improved yield and purity.

### Example 11: Preparation of Osimertinib Methane Sulfonate, Methane Sulfonate Salt of Compound of Formula (1)

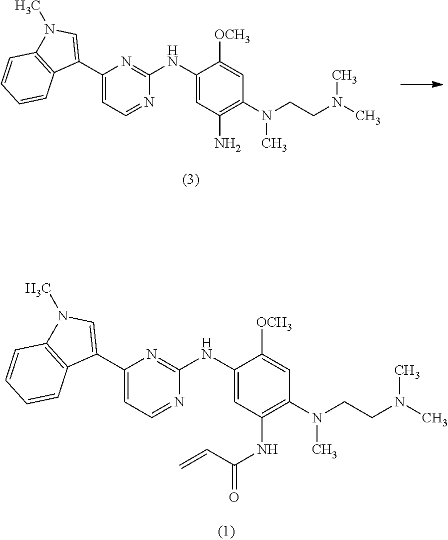 [SMILES: COc1cc(N(C)CCN(C)C)c(N)cc1Nc1nccc(-c2cn(C)c3ccccc23)n1 ; C=CC(=O)Nc1cc(Nc2nccc(-c3cn(C)c4ccccc34)n2)c(OC)cc1N(C)CCN(C)C]

The reaction was done under argon atmosphere. 0.79 g of compound of formula (3) was mixed with 16 ml of 2-methyltetrahydrofuran. To the mixture 2.92 ml of 7% water solution of sodium hydrogen carbonate was added to obtain a solution. To the mixture 0.157 ml of acryloyl chloride was added at 20° C.-25° C. over a period of 1 minute. The mixture was stirred for 30 minutes and 0.585 ml of 7% water solution of sodium hydrogen carbonate followed by 0.026 ml of acryloyl chloride were added. The mixture was stirred for 1.25 hours. The phases were separated, the aqueous phase was discarded and the organic was washed 3× with water, 1× with brine, dried over MgSO₄, filtered and evaporated to afford 0.635 g of compound of formula (1). The rest was dissolved in 8 ml of acetonitrile, immersed in a water bath at 20° C.-25° C. To the mixture 0.105 ml of methanesulfonic acid in 1 ml of acetonitrile was added. The mixture was stirred 12 hours at 20° C.-25° C. to obtain a suspension. The suspension was stirred at 0° C.-5° C. for 30 minutes in ice bath. The solid was filtered off and the filter cake was washed with 2 ml of acetonitrile, suction dried and then dried in vacuum at 40° C. affording 0.683 g (64.4% of theoretical yield) methane sulfonate salt of compound of formula (1), in 94.5% purity.

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

1. A process for preparing a compound of formula (1) or a salt thereof, 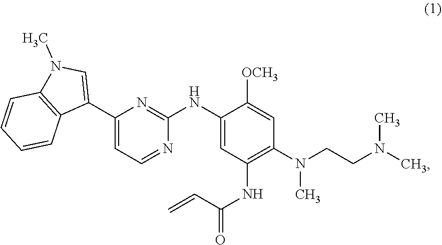 [SMILES: C=CC(=O)Nc1cc(Nc2nccc(-c3cn(C)c4ccccc34)n2)c(OC)cc1N(C)CCN(C)C]
which comprises:
a) reacting a compound of formula (2) with hydrazine or ammonium formate in the presence of a catalyst in a solvent to obtain a compound of formula (3), 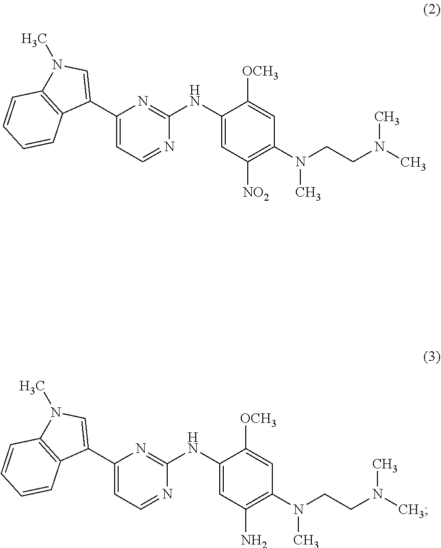 [SMILES: COc1cc(N(C)CCN(C)C)c([N+](=O)[O-])cc1Nc1nccc(-c2cn(C)c3ccccc23)n1 ; COc1cc(N(C)CCN(C)C)c(N)cc1Nc1nccc(-c2cn(C)c3ccccc23)n1]
and
b) transforming the compound of formula (3) into the compound of formula (1).

2. The process according to claim 1 wherein the catalyst is selected from Pd or Pt or Fe or Ru or Rh

3. The process according to claim 2 wherein the catalyst is selected from Pd or Pt or Fe.

4. The process according to claim 3 wherein the catalyst is selected from Pd or Pt.

5. The process according to claim 4 wherein the catalyst is selected from Pd/C or Pt/C.

6. The process according to claim 1 wherein hydrazine is hydrazine hydrate.

7. The process according to claim 1 wherein the molar ratio between the compound of formula (2) and hydrazine or ammonium formate is between 1:3 and 1:20.

8. The process according to claim 7 wherein the ratio is between 1:10 and 1:15.

9. The process according to claim 1 wherein the solvent is selected from 2-methyl tetrahydrofuran or dioxane or tetrahydrofuran or an alcohol or dimethylformamide.

10. The process according to claim 9 wherein the alcohol is selected from methanol or ethanol or propanol.

11. The process according to claim 1 wherein the transforming of the compound of formula (3) into the compound of formula (1) comprises:
a) reacting the compound of formula (3) with a compound of formula (4), 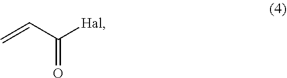
wherein Hal represents Cl or Br; or
b) reacting the compound of formula (3) with a compound of formula (5) to obtain a compound of formula (6) and transforming the compound of formula (6) into the compound of formula (1), 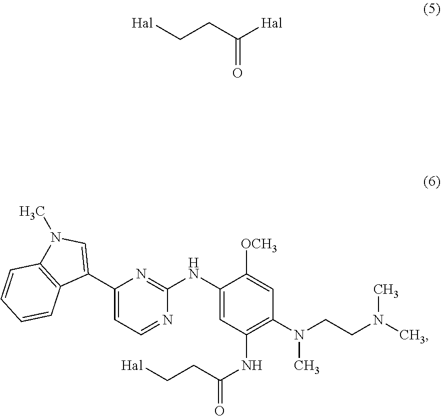
wherein Hal represents Cl or Br.

12. The process according to claim 11 wherein the compound of formula (1) is transformed to a salt.

13. The process according to claim 12 wherein the salt is methane sulfonate salt.
